(19) United States
(12) Patent Application Publication
(10) Pub. No.: US 2009/0197785 A1
(43) Pub. Date: Aug. 6, 2009

(54) STRUCTURED CLEANING COMPOSITIONS

(75) Inventor: John Hawkins, South Staffordshire (GB)

Correspondence Address: PATTERSON, THUENTE, SKAAR & CHRISTENSEN, P.A., 4800 IDS CENTER, 80 SOUTH 8TH STREET, MINNEAPOLIS, MN, 55402-2100, US

(73) Assignee: INNOVATION DELI LIMITED, Boston, Lincolnshire (GB)

(21) Appl. No.: 12/438,250

(22) PCT Filed: Aug. 9, 2007
(86) PCT No.: PCT/GB2007/003023

§ 371 Date: Feb. 20, 2009

(30) Foreign Application Priority Data
Aug. 22, 2006 (GB) 0616624.3
Oct. 5, 2006 (GB) 0619651.3
Dec. 16, 2006 (GB) 0625146.6
Mar. 31, 2007 (GB) 0706353.0
Apr. 26, 2007 (GB) 0708052.6
May 16, 2007 (GB) 0709361.0

(51) Int. Cl. A61K 8/73; A61Q 19/10

(52) U.S. Cl. 510/139

(57) ABSTRACT

A stable, pourable or pasty, homogeneous exfoliant composition having particulate, solid sugar suspended in a saturated aqueous solution of said sugar, and sufficient surfactant to form, in conjunction with said solution, a stable, solid-supporting structured surfactant system.

The present application is a National Phase entry of PCT Application No. PCT/GB2007/003023, filed Aug. 9, 2007, which claims priority from Great Britain Patent Application 0616624.3, filed Aug. 22, 2006, Great Britain Patent Application No. 0619651.3, filed Oct. 5, 2006, Great Britain Patent Application No. 0625146.6, filed Dec. 16, 2006, Great Britain Patent Application No. 0706353.0, filed Mar. 31, 2007, Great Britain Patent Application No. 0708052.6, filed Apr. 26, 2007, and Great Britain Patent Application No. 0709361.0, filed May 16, 2007, the disclosures of which are hereby incorporated by reference herein in their entirety.

### FIELD OF THE INVENTION

The invention relates to structured cleaning compositions for use in personal hygiene. In particular it relates to sugar scrubs, which are useful as skin cleansing products, containing solid particles of sugar as an exfoliant. Such products are gaining increasing popularity.

### BACKGROUND OF THE INVENTION

Wherever the context permits, the term “sugar”, as used herein, embraces all crystalline, highly water-soluble carbohydrates, but will normally be understood to refer in particular to sucrose.

Sugar scrubs typically comprise a paste of sugar, a mild surfactant, fragrance and oil. They are rubbed onto the skin and then washed off, leaving the skin softened and cleansed. Sun and Parr (Toiletries and Cosmetics, Vol. 118, No. 6, June 2003) provide a review of scrub formulations.

Current sugar scrub formulations have a number of disadvantages. They are stiff pastes, which cannot be poured or dispensed from conventional dispensers used for shower gels, shampoos and similar personal cleansing or shower products. On application to the skin much of the solid tends to fall off, creating wastage and mess, and effectively restricting use to the shower cubicle. Moreover, being oil based they require a separate wash with a conventional soap or shower gel, in order to remove the oil. Sun and Parr (vs.) state that sugar scrubs can only be utilised in non-aqueous formulations.

Attempts to solve the problem of dispersing solids in water have generally involved either using gums or other polymeric thickeners to raise the viscosity of the liquid medium, or else forming colloidal dispersions.

Gums and polymeric thickeners, which increase the viscosity of the liquid medium, retard, but do not prevent sedimentation, and at the same time make the composition harder to pour. They do not provide stable suspensions.

Colloidal dispersions contain particles of about 1 micron or smaller, which are prevented from sedimenting by Brownian motion. Such systems are obviously incapable of dispersing relatively coarse particles such as exfoliants.

An alternative to the above methods of suspension would be the use of a structured suspending system. Structured suspending systems depend on the rheological properties of the suspending medium to immobilise the particles, irrespective of size. This requires the suspending medium to exhibit a yield point, which is higher than the sedimenting or creaming force exerted by the suspended particles, but low enough to enable the medium to flow under externally imposed stresses, such as pouring and stirring, like a normal liquid. The structure reforms sufficiently rapidly to prevent sedimentation, once the agitation caused by the external stress has ceased.

The only structured systems, sufficiently effective to have found widespread application, have been based on aqueous surfactant mesophases. Their use has been confined to suspending water-insoluble, or sparingly soluble, solids. Their use for suspending very water-soluble solids, such as sugar has not hitherto been envisaged.

The term “structured system” as used herein means a composition comprising water, surfactant, any structurants, which may be required to impart suspending properties to the surfactant, and optionally other dissolved matter, which together form a mesophase, or a dispersion of a mesophase in a continuous aqueous medium, and which has the ability to immobilise non-colloidal, water-insoluble particles, while the system is at rest, thereby forming a non-sedimenting, fluid or pasty suspension.

Three main types of structured system have been employed in practice, all involving an L_{α}-phase, in which bilayers of surfactant are arranged with the hydrophobic part of the molecule on the interior and the hydrophilic part on the exterior of the bilayer (or vice versa). The bilayers lie side by side, e.g. in a parallel or concentric configuration, sometimes separated by aqueous layers. L_{α}-phases (also known as G-phases) can usually be identified by their characteristic textures under the polarising microscope and/or by x-ray diffraction, which is often able to detect evidence of lamellar symmetry. Such evidence may comprise first, second and sometimes third order peaks with a d-spacing (2π/Q, where Q is the momentum transfer vector) in a simple integral ratio 1:2:3. Other types of symmetry give different ratios, usually non-integral. The d-spacing of the first peak in the series corresponds to the repeat spacing of the bilayer system.

Most surfactants form an L_{α}-phase either at ambient or at some higher temperature when mixed with water in certain specific proportions. However such conventional L_{α}-phases do not usually function as structured suspending systems. Useful quantities of solid render them unpourable and smaller amounts tend to sediment.

The main types of structured system used in practice are based on dispersed lamellar, spherulitic and expanded lamellar phases. Dispersed lamellar phases are two phase systems, in which the surfactant bilayers are arranged as parallel plates to form domains of L_{α}-phase, which are interspersed with an aqueous phase to form an opaque gel-like system. They are described in EP 0 086 614.

Spherulitic phases comprise well-defined spheroidal bodies, usually referred to in the art as spherulites, in which surfactant bilayers are arranged as concentric shells. The spherulites usually have a diameter in the range 0.1 to 15 microns and are dispersed in an aqueous phase in the manner of a classical emulsion, but interacting to form a structured system. Spherulitic systems are described in more detail in EP 0 151 884.

Many structured systems are intermediate between dispersed lamellar and spherulitic, involving both types of structure. Usually systems having a more spherulitic character are preferred because they tend to have lower viscosity. A variant on the spherulitic system comprises prolate or rod shaped bodies sometimes referred to as batonettes. These are normally too viscous to be of practical interest.

Both of the foregoing systems comprise two phases. Their stability depends on the presence of sufficient dispersed phase to pack the system so that the interaction between the spherulites or other dispersed mesophase domains prevents separation. If the amount of dispersed phase is insufficient, e.g. because there is not enough surfactant or because the surfactant is too soluble in the aqueous phase to form sufficient of a mesophase, the system will undergo separation and cannot be used to suspend solids. Such unstable systems are not “structured” for the purpose of this specification.

A third type of structured system comprises an expanded L_{α}-phase. It differs from the other two types of structured system in being essentially a single phase, and from conventional L_{α}-phase in having a wider d-spacing. Conventional L_{α}-phases, which typically contain 60 to 75% by weight surfactant, have a d-spacing of about 4 to 7 nanometers. Attempts to suspend solids in such phases result in stiff pastes which are either non-pourable, unstable or both. Expanded L_{α}-phases with d-spacing greater than 8, e.g. 10 to 15 nanometers, form when electrolyte is added to aqueous surfactants at concentrations just below those required to form a normal L_{α}-phase, particularly to surfactants in the H-phase.

The H-phase comprises surfactant molecules arranged to form cylindrical rods of indefinite length. It exhibits hexagonal symmetry and a distinctive texture under the polarising microscope. Typical H-phases have so high a viscosity that they appear to be curdy solids. H-phases near the lower concentration limit (the L₁/H-phase boundary) may be pourable but have a very high viscosity and often a mucous-like appearance. Such systems tend to form expanded L_{α}-phases particularly readily on addition of sufficient electrolyte.

Expanded L_{α}-phases are described in more detail in EP 0 530 708. In the absence of suspended matter they are generally translucent, unlike dispersed lamellar or spherulitic phases, which are normally opaque. They are optically anisotropic and have shear-dependent viscosity. In this they differ from L₁-phases, which are micellar solutions or microemulsions. L₁-phases are clear, optically isotropic and are usually substantially Newtonian. They are unstructured and cannot suspend solids.

Some L₁-phases exhibit small angle x-ray diffraction spectra, which show evidence of hexagonal symmetry and/or exhibit shear dependent viscosity. Such phases usually have concentrations near the L₁/H-phase boundary and may form expanded L_{α}-phases on addition of electrolyte. However in the absence of any such addition of electrolyte they lack the yield point required to provide suspending properties, and are not, therefore, “structured systems” for the purpose of this specification.

Expanded L_{α}-phases of the above type are usually less robust than spherulitic systems. They are liable to become unstable at low temperatures. Moreover they frequently exhibit a relatively low yield stress, which may limit the maximum size of particle that can be stably suspended.

Most structured surfactants require the presence of a structurant, as well as surfactant and water in order to form structured systems capable of suspending solids. The term “structurant” is used herein to describe any non-surfactant capable, when dissolved in water, of interacting with surfactant to form or enhance (e.g. increase the yield point of) a structured system. It is typically a surfactant-desolubiliser, e.g. an electrolyte. However, certain relatively hydrophobic surfactants such as isopropylamine alkyl benzene sulphonate can form spherulites in water in the absence of electrolyte. Such surfactants are capable of suspending solids in the absence of any structurant, as described in EP 0 414 549.

A problem with the two-phase, especially spherulitic, systems is flocculation of the dispersed surfactant structures. This tends to occur at high surfactant and/or high electrolyte concentration. It can have the effect of making the composition very viscous and/or unstable with the dispersed surfactant separating from the aqueous phase.

Certain amphiphilic polymers have been found to act as deflocculants of structured surfactants. One type of deflocculant polymer exhibits cteniform (comb-shaped) architecture with a hydrophilic backbone and hydrophobic side chains or vice versa. A typical example is a random copolymer of acrylic acid and a fatty alkyl acrylate. Cteniform deflocculants have been described in a large number of patents, for example WO-A-9106622.

A more effective type of deflocculant has surfactant (linear) rather than cteniform architecture, with a hydrophilic polymer group attached at one end to a hydrophobic group. Such deflocculants are typically telomers, formed by telomerising a hydrophilic monomer with a hydrophobic telogen. Examples of surfactant deflocculants include alkyl thiol polyacrylates and alkyl polyglycosides. Surfactant deflocculants are described in more detail in EP 0 623 670.

WO 01/00788 describes the use of carbohydrates such as sugars and alginates as deflocculants in structured surfactant compositions. The latter comprise surfactant, water and electrolyte in proportions adapted to form flocculated two-phase structured surfactant systems in the absence of the carbohydrate.

The use of deflocculant polymers can give rise to syneresis. The spherulitic suspending medium shrinks in volume leaving a clear portion of the continuous phase external to the spherulitic suspending medium. In conventional, aqueous, structured systems, in which the surfactant is normally less dense than the aqueous phase, this usually manifests itself as a clear lower layer (“bottom separation”). Various auxiliary stabilisers have been suggested to inhibit or prevent syneresis or bottom separation of structured surfactant. For example U.S. Pat. No. 5,602,092 has proposed the use of highly cross-linked polyacrylates. WO 01/00779 describes the use as auxiliary stabiliser of non-cross linked polymers with a hydrophilic back bone and sufficient short (e.g. C₁₋₅) hydrocarbon side chains to enhance physical entanglement of the polymer molecules, e.g. polymers of acrylic acid with ethyl acrylate.

Clays such as bentonite or synthetic layered silicates have also been used as auxiliary stabilisers, either alone or in conjunction with polymers.

The use of deflocculant polymers to prepare clear spherulitic or other dispersed L_{α} structured systems, by shrinking the spherulites or other L_{α} domains to a size below the wave length of visible light, has been described in WO 00/63079. The latter also describes the use of sugar to modify the refractive index of the aqueous phase as an alternative means of obtaining clear liquids.

It is known from WO 01/05932 that carbohydrates can interact with surfactants to form suspending structures. Such systems generally exhibit even greater d-spacings than the electrolyte-structured expanded L_{α}-phases, described in EP 0 530 708. The d-spacings of the sugar-structured systems, described in WO 01/05932, are typically greater than 15 nm, and may, for example, be as high as 50 nm. However the specification does not describe systems capable of suspending solid sugar, i.e. suspending systems saturated with dissolved sugar.

### SUMMARY OF THE INVENTION

It has now been discovered that some or all of these problems may be overcome by suspending solid sugar in a pourable aqueous medium. Stable, pourable, homogeneous, exfoliant compositions can be obtained by suspending particulate, solid sugar in a saturated aqueous solution of said sugar and sufficient surfactant to form, in conjunction with said solution, a stable, pourable, solid-supporting structured surfactant system.

The invention, therefore, provides a stable, pourable or pasty, homogeneous exfoliant composition comprising particulate, solid sugar suspended in a saturated aqueous solution of said sugar, and sufficient surfactant to form, in conjunction with said solution, a stable, solid-supporting structured surfactant system.

### DETAILED DESCRIPTION OF THE INVENTION

In the following discussion of the invention, unless stated to the contrary, the disclosure of alternative values for the upper or lower limit of the permitted range of a parameter, coupled with an indication that one of said values is more highly preferred than the other, is to be construed as an implied statement that each intermediate value of said parameter, lying between the more preferred and the less preferred of said alternatives, is itself preferred to said less preferred value and also to each value lying between said less preferred value and said intermediate value.

The sugar is preferably a mono or, more preferably, disaccharide sugar, most preferably sucrose, but could for example be fructose, maltose, glucose or invert sugar. Other sugars, which can be used, include, for example, mannose, ribose, galactose, lactose, allose, altrose, talose, gulose, idose, arabinose, xylose, lyxose, erythrose, threose, acrose, rhamnose, fucose, glyceraldehyde, stachyose, agavose and cellobiose or a tri- or tetra-saccharide.

The surfactant is preferably a mild surfactant of the type commonly used in personal care formulations. It may comprise anionic, amphoteric, zwitterionic, non-ionic and/or cationic surfactants.

A preferred anionic surfactant comprises alkyl ether sulphate, which is preferably the product obtained by ethoxylating a natural fatty or synthetic alcohol with ethylene oxide, optionally stripping any unreacted alcohol, reacting the ethoxylated product with a sulphating agent and neutralising the resulting alkyl ether sulphuric acid with a base. The alcohol has an average of more than 8, preferably more than 10, more preferably more than 12, but less than 30, preferably less than 25, more preferably less than 20, most preferably less than 15 carbon atoms. It is reacted with an average of at least 0.5, preferably more than 1, but less than 60, preferably less than 50, more preferably less than 25, even more preferably less than 15, more preferably still less than 10, most preferably less than 5 ethyleneoxy groups.

Alkyl ether sulphates may also comprise alkyl glyceryl sulphates, and random or block copolymerised alkyl ethoxy/propoxy sulphates.

The anionic surfactant may also comprise, for example, C₁₀₋₂₀ e.g. C₁₂₋₁₈ alkyl sulphate, C₁₀₋₂₀ alkyl benzene sulphonate or a C₈₋₂₀ e.g. C₁₀₋₂₀ aliphatic soap. The soap may be saturated or unsaturated, straight or branched chain. Preferred examples include dodecanoates, myristates, stearates, oleates, linoleates, linolenates, behenates, erucates and palmitates and coconut and tallow soaps. The surfactant may also include other anionic surfactants, such as olefin sulphonates, paraffin sulphonates, taurides, isethionates, ether sulphonates, ether carboxylates, sarcosinates, aliphatic ester sulphonates e.g. alkyl glyceryl sulphonates, sulphosuccinates or sulphosuccinamates.

The cation of any anionic surfactant is typically sodium but may alternatively be potassium, lithium, calcium, magnesium, ammonium, or an alkyl or hydroxyalkyl ammonium having up to 6 aliphatic carbon atoms including ethylammonium, isopropylammonium, monoethanolammonium, diethanolammonium, and triethanolammonium.

Ammonium and ethanolammonium salts are generally more soluble than the sodium salts. Mixtures of the above cations may be used.

The non-ionic surfactants may typically comprise amine oxides, polyglyceryl fatty esters, fatty acid ethoxylates, fatty acid monoalkanolamides, fatty acid dialkanolamides, fatty acid alkanolamide ethoxylates, propylene glycol monoesters, fatty alcohol propoxylates, alcohol ethoxylates, alkyl phenol ethoxylates, fatty amine alkoxylates and fatty acid glyceryl ester ethoxylates. Other non-ionic compounds suitable for inclusion in compositions of the present invention include mixed ethylene oxide/propylene oxide block copolymers, ethylene glycol monoesters, glyceryl esters, ethoxylated glyceryl esters, alkyl polyglycosides, alkyl sugar esters including alkyl sucrose esters and alkyl oligosaccharide esters, sorbitan esters, ethoxylated sorbitan esters, alkyl capped polyvinyl alcohol and alkyl capped polyvinyl pyrrolidone.

Particularly preferred non-ionic surfactants include sugar esters and alkyl polyglycosides, such as C₁₀₋₂₀, preferably _{C10-18}, most preferably C₁₂₋₁₆ alkyl polyglucoside, preferably with a degree of polymerisation between 1.2 and 3.

The surfactant preferably comprises an amphoteric or zwitterionic surfactant. The former preferably comprises so-called imidazoline betaines, which are also called amphoacetates, and were traditionally ascribed the zwitterionic formula:

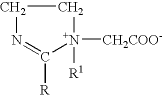

because they are obtained by reacting sodium chloracetate with an imidazoline. It has been shown, however, that they are actually present, at least predominantly, as the corresponding, amphoteric, linear amidoamines:

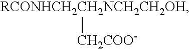

which are usually obtained commercially in admixture with the dicarboxymethylated form:

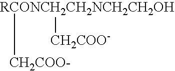

R preferably has at least 8, more preferably at least 10 carbon atoms but less than 25, more preferably less than 22, even more preferably less than 20, most preferably less than 18. Typically R represents a mixture of alkyl and alkenyl groups, obtained, for example, from coconut or palm oil, and having sizes ranging from 8 to 18 carbon atoms, with 12 predominating, or a fraction of such a feedstock, such as lauryl (>90% C₁₂). R may alternatively be a residue derived from a terpene, such as an adduct of acrylic acid with myrcene or α-terpinene.

The zwitterionic surfactant is preferably a betaine, phosphobetaine or most preferably a sulphobetaine, which typically has the formula R″R′₂ NCH₂XOH, where X is CO, PO or preferably SO₂, R′ is an aliphatic group having 1 to 4 carbon atoms and R″ is an aliphatic group having from 8 to 25 carbon atoms, preferably a straight or branched chain alkyl or alkenyl group, or more preferably a group of the formula RCONR′(CH₂)ₙ, where R and R′ have the same significance as before, and n is an integer from 2 to 4.

We prefer that R′ is a methyl, carboxymethyl, ethyl, hydroxyethyl, carboxyethyl, propyl, isopropyl, hydroxypropyl, carboxypropyl, butyl, isobutyl or hydroxybutyl group.

The surfactant may comprise cationic surfactants such as fatty alkyl trimethylammonium or benzalkonium salts, amidoamines or imidazolines.

The surfactants preferably have a mean HLB greater than 5.5, more preferably greater than 6.5, even more preferably greater than 8, more preferably still, greater than 8.5, most preferably greater than 9. Difficulty may be encountered obtaining stable suspensions with surfactant systems having a mean HLB greater than 45. Preferably the HLB is less than 40, more preferably less than 35, even more preferably less than 20, most preferably less than 15.

Where it is desired to use a surfactant of relatively high HLB, such as an ether sulphate it is possible to improve the stability by adding an electrolyte, as an auxiliary structurant. The electrolyte is typically sodium chloride, but could, for example, alternatively or additionally, be or comprise, sodium carbonate, potassium chloride, sodium phosphate, sodium citrate or any other surfactant desolubilising electrolyte. The proportion of electrolyte required generally depends on the amount and HLB of the surfactant, but typically lies within the range 0 to 15% by weight, based on the weight of the composition, preferably less than 12%, most preferably less than 11%. Higher HLB surfactants require higher levels of electrolyte.

Alternatively, or additionally the high HLB surfactant may be used in conjunction with a surfactant of lower HLB, such as oleic acid or more preferably oleyl alcohol or a sorbitan or glyceryl ester such as sorbitan or glyceryl mono oleate, so that the mean value of the surfactant mixture lies within the preferred range.

The aqueous structured systems, formed by the interaction of surfactants with carbohydrates, according to our invention typically comprise systems, which are either spherulitic or expanded L_{α}-phase. They include systems having a repeat spacing greater than 8, preferably greater than 10, more preferably greater than 20, most preferably greater than 30 nm, up to or above 60 nm.

We generally prefer that the surfactant is present in a total concentration greater than 4% by weight, based on the total weight of the composition, more preferably greater than 5%, still more preferably greater than 10%, most preferably greater than 12%. Preferably the surfactant concentration is less than 25%, more preferably less than 18%, most preferably less than 16% by weight.

If a sufficiently stable system cannot be found within this range, electrolyte may be added incrementally to the composition, until a stable spherulitic or expanded lamellar phase is obtained. Generally the presence of a spherulitic phase can be detected by measuring the conductivity as the electrolyte is added, as described, for instance in EP 0 151 884. Where the conductivity falls and passes through a trough a stable spherulitic phase may be found, typically at concentrations within ±5%, by weight based on the weight of the surfactant, of that corresponding to the minimum value of the trough, preferably ±2%, most preferably ±1%.

For the purpose of this specification “stable” indicates that the suspended solid does not sediment after six months at room temperature. The term does not exclude up to 10% by volume of bottom separation, which may be observed, e.g. when using high HLB surfactants stabilised with electrolyte, and may even be an advantage in dispensers of the shower-gel type in which the composition is dispensed from the bottom of the container. It may inhibit accumulation of solids in the container neck.

Bottom separation is most commonly observed in systems containing electrolyte. It can generally be avoided by increasing the total concentration of surfactant. This generally results in increased viscosity, and is therefore most suitable for pasty products supplied in tubs, like conventional body scrubs. Electrolyte may reduce stability at higher temperatures, e.g. above 40° C. Where this is a problem, , it is preferred to replace any electrolyte, wholly or in part by a low HLB surfactant, preferably a non-ionic surfactant having an HLB below 9, more preferably below 8, still more preferably below 6, most preferably below 5. Preferred low HLB surfactants include sorbitan oleate, glyceryl oleate and oleyl alcohol. As in the case of electrolyte the required amount of low HLB surfactant may be routinely determined by observing the conductivity of the system while adding increments of the surfactant, and noting the position of the conductivity trough.

The discussion is based on the assumption that the structure is lamellar or spherulitic. We do not, however, intend to exclude the possibility that the system may comprise non-lamellar components.

The levels of carbohydrate may be sufficiently high to inhibit microbiological growth in the medium and sufficient to act as an effective biodegradable, non-allergenic preservative for the composition.

Preferably the surfactant is stirred into the saturated carbohydrate solution, and if a sufficiently stable suspending system is not obtained, electrolyte, or low HLB surfactant is added in small increments until an acceptable yield point is achieved.

The solid exfoliant is present in total concentrations greater than saturation at ambient temperature. The composition generally comprises suspended solid sugar in amounts, at room temperature, greater than 3%, preferably greater than 10%, more preferably greater than 15%, even more preferably greater than 20%, most preferably greater than 25% by weight, based on the weight of the composition. Amounts of suspended solid greater than 50% by weight are usually undesirably viscous. We prefer that the suspended solid should be less than 40% by weight, more preferably less than 35%

The suspended solid sugar typically has a relatively coarse granular texture, with a mean particle size greater than 100 microns, preferably greater than 500 microns, more preferably greater than 1 mm, most preferably greater than 1.5 mm, but less than 5 mm, preferably less than 3 mm, most preferably less than 2 mm.

According to a specific embodiment the invention provides lip, tongue or mouth exfoliants which consist of food-acceptable ingredients. Typically finer grades of sugar, e.g. caster sugar, are preferred for such applications.

Usually the total concentration of sugar is greater than 45%, preferably greater than 50%, most preferably greater than 60%, but less than 80%, preferably less than 75%, most preferably less than 70%, by weight, based on total weight of the composition.

The product may optionally contain other common ingredients of personal cleansers, such as buffers, antioxidants, glycerol, essential oils, fragrances, pigments, dyes, pearlisers, emollients, antiseptics and topical medicaments.

Buffers may be required to obtain optimum pH for stability of the ingredients and/or skin sensitivity. We prefer that the pH is less than 8, more preferably less than 7, most preferably less than 5.8, but more than 4, more preferably more than 5, most preferably more than 5.2. Suitable buffers, depending on the desired pH include citrate (e.g. trisodium citrate/citric acid), acetate, phosphate and tartrate buffers.

The product may a readily pourable fluid, or a paste. Typically the viscosity at 21 reciprocal seconds shear is greater than 0.1 Pa s, more preferably greater than 1 Pa s, most preferably greater than 5 Pa s, but less than 25 Pa s, more preferably less than 20 Pa s, most preferably less than 15 Pa s.

The invention will be illustrated by the following examples, in which all proportions are expressed as % by weight based on the total weight of the composition unless stated to the contrary.

Each of the following examples I to IV was a stable, pourable spherulitic suspension with a pleasant feel when rubbed on the skin and an effective cleansing and skin softening action. In each case the balance was water, to which ingredients were added cold, with stirring, in the stated order.

### Example I

65% soft brown sugar

6.75% sodium C₁₂₋₁₄ alkyl 2-mole ethoxy sulphate

2.1% coconut diethanolamide,

2.6% sodium chloride.

### Example II

65% white sugar

6.7% sodium lauryl 2 mole ethoxy sulphate

2.2% coconut diethanolamide

1.0% oleic acid

0.2% peppermint oil

### Example III

70% white sugar

6% isopropylamonium dodecylbenzene sulphonate

### Example IV

70% white sugar

6% coconut diethanolamide.

### Example V

The following ingredients were mixed in the order given by cold stirring:

| | |
|---|---|
| 67% w/w aqueous sucrose solution | 619.6 g |
| Dry sodium chloride | 24.9 g |
| Coconut diethanolamide | 22.4 g |
| 70% aqueous sodium lauryl 2 mole ethoxy sulphate | 96.5 g |
| Peppermint oil | 1.8 g |
| Granulated white sugar | 234.8 g |

The composition was spherulitic under the polarising microscope and was non-sedimenting after two months at 40° C., ambient room temperature and 40° C. A small bottom separation of 5% by volume appeared after a few days at 40° C. and about two weeks at room temperature. A smaller separation was observed at 4° C.

### Example VI

The following ingredients were mixed in the order given by cold stirring:

| | |
|---|---|
| 67% w/w aqueous sucrose solution | 286.3 g |
| Dry sodium chloride | 10.0 g |
| 45% w/w aqueous cocamidopropyl hydroxysultaine | 25.0 g |
| 70% w/w aqueous sodium lauryl 2 mole ethoxy sulphate | 48.2 g |
| Granulated white sugar | 133.0 g |

The composition was spherulitic under the polarising microscope and was non-sedimenting and showed no bottom separation.

### Examples VII-X

The following formulations were prepared without electrolyte. They were non-sedimenting. Example X was made using caster sugar and was suitable for lip, tongue and mouth cleansing.

| | EXAMPLE | | | |
|---|---|---|---|---|
| | VII | VIII | IX | X |
| Sucrose distearate | 1.75 | 1.15 | 1.5 | 1.15 |
| Sucrose monostearate | 1.75 | 1.15 | 1.5 | 1.15 |
| Coconut diethanolamide | 3.5 | 4.65 | | |
| C8/10 alkyl polyglycoside | | | 1.0 | |
| Sorbitan monolaurate 5 mole ethoxylate | | | 3.0 | 4.65 |
| Sucrose | 70.0 | 70.0 | 70.0 | 65.0 |
| Chocolate powder | | | | 5.0 |
| Peppermint oil | | | | 0.125 |

### Example XI

| | |
|---|---|
| 67% aqueous sucrose | 500 g |
| Sodium chloride | 20 g |
| 45% w/w aqueous cocamidopropyl hydroxysultaine | 55.5 g |
| Peppermint oil | 2 g |
| Granulated white sugar | 315 g |
| 70% w/w aqueous sodium lauryl 2 mole ethoxy sulphate | 107 g |

The above ingredients were mixed in the order shown with gentle stirring at room temperature. Before adding the ether sulphate, the mixture was allowed to deaerate, while being gently stirred. The product was non-sedimenting, and was fully stable after six weeks at 40° C., but showed 4% bottom separation after 2 days at 50° C.

### Example XII

In order to avoid bottom separation, Example XI was repeated using higher active concentrations, as follows

| | |
|---|---|
| 67% aqueous sucrose | 355 g |
| Sodium chloride | 20 g |
| 45% w/w aqueous cocamidopropyl hydroxysultaine | 72 g |
| Peppermint oil | 2 g |
| Granulated white sugar | 412 g |
| 70% w/w aqueous sodium lauryl 2 mole ethoxy sulphate | 139 g |

The composition gave no bottom separation after 5 days at 50° C.

### Example XIII

Example XI was reformulated without salt, using a low HLB surfactant.

| | |
|---|---|
| 45% w/w aqueous cocamidopropyl hydroxysultaine | 11.1 g |
| 70% w/w aqueous sodium lauryl 2 mole ethoxy sulphate | 21.4 g |
| Sorbitan mono oleate (HLB 4.3) | 6.0 g |
| 67% aqueous sucrose | 100 g |
| Soft brown sugar | 63 g |

The composition was fully stable after 2 weeks at 40° C.

### Example XIV

| | |
|---|---|
| Sodium lauryl 2 mole ethoxy sulphate | 9.75 |
| Cocamidopropyl sulphobetaine | 3.25 |
| Sodium chloride | 2.00 |
| Sucrose | 65.00 |
| Peppermint oil | 0.20 |

The formulation was a viscous paste, which was stable at 45° C. after 3 months.

### Examples XV-XVII

| | Example | | |
|---|---|---|---|
| | XV | XVI | XVII |
| Oleyl alcohol | 3.25 | 1.84 | 1.74 |
| C12/16 alkyl polyglucoside | 13.00 | | |
| Sodium lauryl 2 mole ethoxy sulphate | | 8.35 | 9.67 |
| Cocamidopropyl sulphobetaine | | 2.77 | 3.22 |
| Sucrose | 65.00 | 64.90 | 62.50 |

Each of the above examples XV -XVII was stable for three months at 45° C.

### Examples XVIII and XIX

Example

| | XVIII | XIX |
|---|---|---|
| Sodium lauryl 2 mole ethoxy sulphate | 9.65 | 9.68 |
| Cocamidopropyl hydroxysultone | 3.21 | 3.11 |
| Sucrose | 62.34 | 62.48 |
| Sodium chloride | 1.98 | |
| Oleyl alcohol | | 1.74 |
| Soya oil | 2.98 | 2.98 |
| Shea butter | 0.25 | 0.25 |
| Perfume | 1.00 | 1.00 |

### Example XX

It was desired to prepare a composition based on lauryl polyglucoside and sucrose. The lauryl polyglucoside was found to give separation when structured with sugar alone. A series of systems containing increments of oleyl alcohol were therefore prepared and the conductivity measured. At 15% by weight total surfactant, a conductivity trough was found with a minimum at an oleyl alcohol concentration of 14% by weight of the total surfactant. Stable suspending systems were obtained at concentrations between 9 and 20% by weight oleyl alcohol, based on total surfactant. Based on the above observation, the following stable composition was prepared.

A blend (A) comprised 29.6 g soya bean oil, 9.6 g perfume and 0.4 g tocopherol. The ingredients were gently stirred together.

A blend (B) comprised 3.7 g citric acid monohydrate, 2.2 g trisodium citrate dihydrate, 2.0 g titanium oxide and 24 g deionised water, which was stirred gently until needed.

280.0 g of 50% active lauryl polyglucoside (“PLANTACARE”® 1200 UP), 19 g oleyl alcohol, 39.6 g of blend A, 31.9 g of dispersion B and 2.5 g of molten shea butter at 40° C., were charged successively to a 600 ml beaker and stirred until homogeneous. 577.0 g of white granulated sugar and 50.0 g golden organic sugar were added and stirred into the mixture. The product was a white paste with a pH of 5.5 and a conductivity of 20 ms. It was stable after three months at 45° C., laboratory ambient and 5° C.

### Examples XXI-XXV

All these examples were soft pourable thin pastes buffered to pH 5.5 (skin pH). They spread easily on the skin and may be rubbed in to give a gentle exfoliating action. They are readily washed off with warm water and they leave the skin soft, clean, fragranced, and moisturized. The samples were stable for 3 months at 45 Degrees Centigrade, Room Temperature, and 3 degrees Centigrade

| | Example | | | | |
|---|---|---|---|---|---|
| | XXI | XXII | XXIII | XXIV | XXV |
| Sodium lauryl ether | 9.8 | 9.0 | 7.3 | 9.6 | |
| (2EO) sulphate | | | | | |
| Cocamidopropylbetaine | | 3.0 | 1.83 | | |
| Cocamidopropyl- | 3.3 | | | | |
| sulphobetaine | | | | | |
| Lauryl Glucoside | | | 1.83 | 2.4 | 11.0 |
| Oleyl Alcohol | | | | 0.5 | |
| White Granulated Sugar | 58.3 | 57.0 | 58.0 | 58.0 | 58.0 |
| Organic Cane Sugar | 5.0 | 4.0 | 5.0 | 5.0 | 5.0 |
| Refined Soybean Oil | 3.0 | 5.0 | 5.0 | 5.0 | 5.0 |
| Refined Shea Butter | 0.25 | 0.25 | 0.25 | 0.25 | 0.25 |
| Trisodium Citrate | 0.15 | 0.15 | 0.15 | 0.15 | 0.15 |
| Dihydrate | | | | | |
| Citric Acid Monohydrate | 0.25 | 0.25 | 0.25 | 0.25 | 0.25 |
| Sodium Chloride | | 2.0 | 1.75 | 2.5 | 8.0 |
| Perfume(Shea Butter) | 1.0 | 1.0 | 1.0 | 1.0 | 1.0 |
| Total Solids | 81.05 | 81.65 | 82.36 | 84.65 | 88.65 |
| Conductivity | 240 | 230 | 140 | 80 | 250 |
| microseimens | | | | | |

### Example XXVI

| | |
|---|---|
| Oleyl Alcohol | 1.91 |
| Lauryl Glucoside | 13.96 |
| Baobab Oil | 2.00 |
| Perfume (Moroccan Brown Sugar 90209) | 0.22 |
| White Granulated Sugar | 56.32 |
| Soft Brown Sugar | 6.18 |
| Citric Acid | 0.4 |
| Trisodium citrate dihydrate | 0.2 |

The scrub was an ‘all natural’ soft paste that spread easily on the skin and was rubbed in to give a gentle exfoliating action. It was readily washed off with water to leave skin soft, clean, fragranced, and moisturised.

The sample was stable for 3 months at 45° C., laboratory ambient temperature, and 3° C.

1-14. (canceled)

15. A stable, pourable or pasty, homogeneous exfoliant composition comprising particulate, solid sugar characterised in that said sugar is suspended in a saturated aqueous solution of said sugar, and sufficient surfactant to form, in conjunction with said solution, a stable, solid-supporting structured surfactant system.

16. A composition according to claim 15 characterised in that the sugar is a monosaccharide or disaccharide.

17. A composition according to claim 16 characterised in that the sugar is sucrose.

18. A composition according to claim 15 characterised in that the surfactant comprises an anionic surfactant

19. A composition according to claim 16 characterised in that the surfactant comprises an anionic surfactant.

20. A composition according to claim 18 characterised in that the surfactant comprises an alkyl ether sulphate.

21. A composition according to claim 15 characterised in that the surfactant comprises a non-ionic surfactant.

22. A composition according to claim 15 characterised in that the surfactant comprises an alkanolamide.

23. A composition according to claim 15 characterised in that the surfactant comprises a sugar ester or alkyl polyglycoside

24. A composition according to claim 15 characterised in that the surfactant comprises an amphoteric or zwitterionic surfactant.

25. A composition according to claim 15 characterised in that the surfactant comprises a betaine or sulphobetaine

26. A composition according to claim 15 characterised in that the solution comprises an electrolyte as an auxiliary structurant

27. A composition according to claim 26 characterised in that the electrolyte is sodium chloride

28. A composition according to claim 15 characterised in that the surfactant comprises a non-ionic surfactant having an HLB below 9

29. A composition according to claim 28 characterised in that said non-ionic surfactant is oleyl alcohol, sorbitan mono oleate or glyceryl mono oleate

30. A composition according to claim 15, wherein the concentration of sugar is between about 45 wt % and about 80 wt %.

31. A composition according to claim 15, wherein the sugar has a mean particle size of about 100 micron to about 5 mm.

32. A composition according to claim 15, wherein the surfactant is present in the composition in an amount of between about 4 wt % and about 25 wt %.

33. A composition according to claim 15, wherein the pH of the composition is between about 4 and about 8.

34. A composition according to claim 15, further comprising one or more of a personal cleanser, a buffers, an antioxidant, glycerol, an essential oil, a fragrance, a pigment, a dye, a pearliser, an emollient, an antiseptic and a topical medicament.
